(19) United States
(12) Patent Application Publication
(10) Pub. No.: US 2024/0101497 A1
(43) Pub. Date: Mar. 28, 2024

(54) PROCESS FOR PRODUCING LOW, NEUTRAL, AND/OR NEGATIVE CARBON INTENSITY ETHYLENE UTILIZING HYDROGEN PRODUCED THROUGH REFORMING OF HYDROCARBONS AND/OR ELECTROLYSIS OF WATER FROM BIOMASS ENERGY

(71) Applicant: Kraken Technology Holdings, LLC, Houston, TX (US)

(72) Inventors: Christopher Michael MILLER, Richmond, TX (US); Gregory David SMITH, Fulshear, TX (US); Anna Louise BUCKLEY, Houston, TX (US); Byron Gladus BEST, III, Katy, TX (US); Bengt Arne JARLSJO, Houston, TX (US); Dainel Joseph SHAPIRO, Houston, TX (US)

(21) Appl. No.: 18/473,801

(22) Filed: Sep. 25, 2023

Related U.S. Application Data
(63) Continuation-in-part of application No. 18/117,606, filed on Mar. 6, 2023.
Application No. 18/117,606 is a continuation-in-part of application No. 18/471,768, filed on Sep. 21, 2023.
(60) Provisional application No. 63/578,014, filed on Aug. 22, 2023.
(60) Provisional application No. 63/409,331, filed on Sep. 23, 2022.
(60) Provisional application No. 63/451,940, filed on Mar. 14, 2023.

(51) Int. Cl. C07C 4/04; C01B 3/38; C07C 7/04; C25B 1/04; C25B 9/19; C25B 13/08; C25B 15/08

(52) U.S. Cl. CPC C07C 4/04; C01B 3/38; C07C 7/04; C25B 1/04; C25B 9/19; C25B 13/08; C25B 15/081; C01B 2203/0233; C01B 2203/0244; C01B 2203/062; C01B 2203/0816; C01B 2203/1241

(57) ABSTRACT

A method for producing an olefin product, including the steps of converting a hydrocarbon feedstock to an unsaturated hydrocarbon stream through a steam cracking process in an olefins production plant; combusting hydrogen to provide at least some of the heating duty to the steam cracking process, wherein the hydrogen has a carbon intensity less than about 1.0 kg CO₂e/kg H₂, wherein the hydrogen is produced using a hydrogen production process; providing at least some of the required energy for the hydrogen production process from a biomass power plant; and processing the unsaturated hydrocarbon stream to recover the olefin product. The olefin product may comprise ethylene having a well-to-gate carbon intensity less than about 0.6 kg CO₂e/kg C₂H₄, or may comprise propylene having a well-to-gate carbon intensity less than about 0.6 kg CO₂e/kg C₃H₆.

### CROSS-REFERENCE TO RELATED APPLICATION

This application claims the benefit of U.S. Prov. App. Nos. 63/578,014 (filed Aug. 22, 2023), 63/409,331 (filed Sep. 23, 2022), and 63/451,940 (filed Mar. 14, 2023); and is a continuation-in-part of U.S. application Ser. No. 18/117,606 (fil. March 6, 2023) and Ser. No. 18/471,768 (filed Sep. 21, 2023); each of which is incorporated by reference herein in its entirety.

### BACKGROUND

The present invention relates to a method and process for producing an ethylene product having reduced carbon intensity. More specifically, the method includes the production of ethylene by a steam cracking process, where the heating duty for the steam cracking process is provided by the combustion of low, neutral, and/or negative carbon intensity hydrogen to reduce the carbon intensity of the ethylene product.

Ethylene (C₂H₄), an important petrochemical precursor, can be used as a feedstock to manufacture various specialty chemicals and plastics, such as polyethylene, ethylene oxide, ethylene dichloride, ethylbenzene, and vinyl acetate, which are used in the packaging, transportation, construction, industrial, and consumer markets.

The global ethylene market is estimated to grow at a compounded annual growth rate between 4 to 6% by 2035 largely due to the increased demand for polyethylene. Global ethylene production in 2021 was nearly 160 million metric tons per year and is expected to grow to over 260 million metric tons per year by 2035.

Currently, the petrochemical industry accounts for approximately 6% of the global greenhouse gas (GHG) emissions with ethylene production accounting for approximately 0.8% of the total global carbon emissions, which equates to approximately 260 million metric tons per year of CO₂ emissions. As the demand for ethylene production increases, the carbon dioxide emissions associated with the steam cracking process will also continue to increase.

Commercial scale ethylene production utilizes a highly endothermic steam cracking process that requires a significant amount of external heating duty, which is typically provided by the combustion of a hydrocarbon fuel in the steam cracking furnace.

In order to achieve a net reduction of carbon emissions, ethylene must be produced with low carbon technologies and/or low carbon feedstocks. Currently available options for producing reduced carbon intensity ethylene include: (1) reducing the fired duty of the steam cracking furnace by optimizing the energy input and heat integration of the process; (2) electrification, or use of electric motors to drive the main compressors; (3) using low carbon intensity electricity to provide the external heating duty to the steam cracking process; (4) capturing the CO₂ emissions associated with the combustion of hydrocarbon fuels from the steam cracking furnaces and/or boilers; (5) increasing the hydrogen content in the steam cracking furnace fuel gas; and (6) reducing the upstream emissions associated with the production of the steam cracking process feedstocks (e.g., ethane, propane, butane, naphtha, and/or gas oil feeds).

Ethylene carbon intensity may be evaluated using a life cycle analysis methodology such as Argonne National Laboratory's Greenhouse Gases, Regulated Emissions, and Energy Use in Technologies Model (GREET). The term “carbon intensity” refers to a measure of the amount of equivalent carbon dioxide (CO₂e) emitted to produce a specified amount of a product, such as ethylene. CO₂e is a common unit used to sum various greenhouse gases based on their global warming potential (GWP). Ethylene carbon intensity is frequently rated in kilograms of equivalent carbon dioxide per kilogram of ethylene (kg CO₂e/kg C2H4).

Ethylene produced by a conventional steam cracking process will normally result in a well-to-gate carbon intensity between 1.0 and 1.6 kg CO₂e/kg C₂H₄. Approximately 80% of the onsite CO₂emissions are generated from the combustion of a hydrocarbon fuel to provide the heating duty of the steam cracking process. Ethylene with a carbon intensity closer to 1.0 kg CO₂e/kg C₂H₄ is often produced from a steam cracking process that uses an ethylene off gas stream comprised mostly of hydrogen and methane, produced internal to the ethylene production process as the fuel to the steam cracking furnaces. Whereas ethylene with a carbon intensity closer to 1.6 kg CO₂e/kg C₂H₄, is often produced using imported hydrocarbon fuel to the cracking furnaces.

Conventional steam cracking is a highly endothermic process that requires heat and energy, both of which contribute to the overall CO₂e emissions of the steam cracking process. Sources of CO₂e emissions in the production of ethylene comprise: (1) upstream emissions associated with the steam cracker feed; (2) emissions associated with the production of imported power; and (3) and uncaptured CO₂e emissions associated with the steam cracking process.

The upstream emissions associated with the steam cracker feed (e.g., methane leakage from natural gas transmission, emissions associated with NGL, naphtha, and/or gas oil production), are inherent to the process and can only be reduced, not fully eliminated, by utilizing low carbon intensity feeds.

The emissions associated with the production of imported power can be reduced or eliminated by utilizing a source of low or neutral carbon intensity power (e.g., solar, wind, geothermal, hydro, or nuclear power).

The uncaptured CO₂e emissions associated with the steam cracking process can be reduced or eliminated with the use of a post-combustion capture (PCC) unit, an electric furnace, or increasing the hydrogen content in the steam cracking furnace fuel gas.

Even with the use of low carbon intensity feeds, carbon neutral power, and eliminating the uncaptured, onsite CO₂e emissions associated with the steam cracking furnace, the lowest practically achievable ethylene product carbon intensity is still greater than 0.6 kg CO₂e/kg C₂H₄.

Producing ethylene in accordance with the present invention preferably results in a product carbon intensity preferably less than about 0.6 kg CO₂e/kg C₂H₄, more preferably less than about 0.4 kg CO₂e/kg C₂H₄, and most preferably less than about 0.0 kg CO₂e/kg C₂H₄.

### SUMMARY OF THE INVENTION

A method for producing an ethylene product having a well-to-gate carbon intensity preferably less than about 0.6 kg CO₂e/kg C₂H₄, more preferably less than about 0.4 kg CO₂e/kg C₂H₄, and most preferably less than about 0.0 kg CO₂e/kg C₂H₄, is provided. Also included is a method for producing a propylene product having a well-to-gate carbon intensity preferably less than about 0.6 kg CO₂e/kg C₃H₆, more preferably less than about 0.4 kg CO₂e/kg C₃H₆, and most preferably less than about 0.0 kg CO₂e/kg C₃H₆.

The method comprises the steps of converting a hydrocarbon feedstock to an unsaturated hydrocarbon stream through a steam cracking process in an olefins production plant; combusting hydrogen to provide at least some of the heating duty to the steam cracking process, wherein the hydrogen has a carbon intensity preferably less than about 1.0 kg CO₂e/kg H₂, more preferably less than less than about 0.45 kg CO₂e/kg H₂, and most preferably less than about 0.0 kg CO₂e/kg H₂, wherein the hydrogen is produced using a hydrogen production process; providing at least some, and preferably substantially all, of the required energy for the hydrogen production process from a biomass power plant; and processing the unsaturated hydrocarbon stream to recover the olefin product.

The hydrocarbon feedstock for the olefins production plant is selected from ethane, propane, butane, naphtha, gas oils, or a combination thereof. One or more gas streams containing carbon dioxide from the biomass power plant may be processed in one or more carbon capture units to reduce CO₂e emissions. The hydrogen production process may comprise converting a hydrocarbon feedstock to hydrogen through a reforming process, or may comprise the electrolysis of water, or both. At least some, and preferably substantially all, of the required electricity and steam energy for olefins production plant is provided from the biomass power plant.

### DESCRIPTION OF FIGURES

The features and advantages of the present invention will be more clearly understood from the following description taken in conjunction with the accompanying drawings in which:

FIG. 1 depicts a process flow diagram for the production of low, neutral, and/or negative carbon intensity ethylene, wherein the heating duty for the steam cracking process is provided by the combustion of low, neutral, and/or negative carbon intensity hydrogen produced by a reforming process.

FIG. 2 depicts a process flow diagram for the production of low, neutral, and/or negative carbon intensity ethylene, wherein the heating duty for the steam cracking process is provided by the combustion of low, neutral, and/or negative carbon intensity hydrogen produced by an electrolysis process.

FIG. 3 depicts a process flow diagram for the process of FIG. 1, in which the reforming process is illustrated as an auto-thermal reformer wherein methane from the steam cracking process is recycled as feed to the reformer.

FIG. 4 depicts a process flow diagram for the process of FIG. 2, in which hydrogen, recovered from the ethylene off gas, is recycled as fuel to the stream cracking furnace.

### DETAILED DESCRIPTION OF THE INVENTION

The present invention relates to a method and a process for producing ethylene in which at least some of the heating duty for the steam cracking process is provided by the combustion of low, neutral, and/or negative carbon intensity hydrogen to reduce the carbon intensity of the ethylene product to preferably less than 0.6 kg CO₂e/kg C₂H₄, more preferably less than 0.4 kg CO₂e/kg C₂H₄, and most preferably less than 0.0 kg CO₂e/kg C₂H₄.

In all embodiments of the present invention described herein, the low, neutral, and/or negative carbon intensity hydrogen may be produced according to the teachings of commonly owned U.S. application Ser. No. 18/117,606 (filed Mar. 6, 2023), which is incorporated by reference herein in its entirety. As discussed therein, the energy for the hydrogen production process is provided by the combustion or gasification of various forms of biomass to reduce the carbon intensity of the hydrogen product to preferably less than about 1.0 kg CO₂e/kg H₂, more preferably less than about 0.45 kg CO₂e/kg H₂, and most preferably less than about 0.0 kg CO₂e/kg H₂.

In all embodiments of the present invention described herein, the low, neutral, and/or negative carbon intensity hydrogen may also be produced according to the teachings of commonly owned U.S. application Ser. No. 18/471,768 (filed Sep. 21, 2023), which is incorporated by reference herein in its entirety. As discussed therein, the energy to produce hydrogen through electrolysis is provided by the combustion or gasification of various forms of biomass to reduce the carbon intensity of the hydrogen product to preferably less than about 0.45 kg CO₂e/kg H₂ and more preferably less than about 0.0 kg CO₂e/kg H₂.

Furthermore, in all embodiments described herein, the biomass feed to the biomass power plant can be a variety of fuel mixes including but not limited to: woody biomass, municipal solid waste (MSW), sorted MSW, food waste, agriculture waste, landfill diversion, hurricane and construction debris, industrial processing biomass waste, and renewable natural gas. A person having ordinary skill in the art will appreciate that fossil fuels, including but not limited to natural gas and/or fuel gas (i.e. propane and butanes), can also be co-fired with the biomass as required or desired.

Furthermore, in all embodiments described herein, the biomass power plant is selected from a Rankine cycle, or a Brayton cycle, or an integrated gasification combined cycle (IGCC) consisting of both a Rankine cycle and Brayton cycle.

Where the biomass power plant configuration comprises a Rankine cycle, a biomass boiler produces heat from the direct combustion of biomass to generate steam for the Rankine cycle. In this configuration, the biomass boiler can either be a traditional air fired boiler or oxy-fired boiler. In instances where the boiler is oxy-fired, the oxygen can be supplied by the electrolysis unit (if applicable), an air separation unit, or otherwise supplied by a third-party. Furthermore, where the biomass power plant configuration comprises a Brayton cycle, the gasification of biomass produces a syngas product, with or without steam generation, that is purified before being combusted in the Brayton cycle. The exhaust heat from the Brayton cycle can be used to generate steam for a Rankine cycle.

In all embodiments of the present invention described herein, carbon negative energy from the biomass power plant can be provided as: (1) electrical power, (2) process or dilution steam, (3) thermal energy in the form of steam, and/or (4) steam drive energy to power mechanical drives (e.g., turbines) of the compressors, pumps, and/or fans.

In all embodiments described herein, the electricity generated by the biomass power plant can equal, but does not have to equal, the total amount of electricity required for producing hydrogen either by a reforming process or by electrolysis. A person having ordinary skill in the art will understand that, in most applications, the biomass power plant can be sized to generate adequate carbon negative energy to offset enough import grid power to achieve a targeted hydrogen carbon intensity.

Furthermore, in all embodiments described herein, the carbon negative energy produced from the biomass power plant may be combined with higher carbon intensity energy (e.g., local grid electricity and/or electricity produced from a gas turbine) to thereby achieve a targeted hydrogen carbon intensity.

Furthermore, in all embodiments of the present invention described herein, the biogenic carbon dioxide produced from the combustion or gasification of biomass may be separated in a carbon capture unit for permanent sequestration.

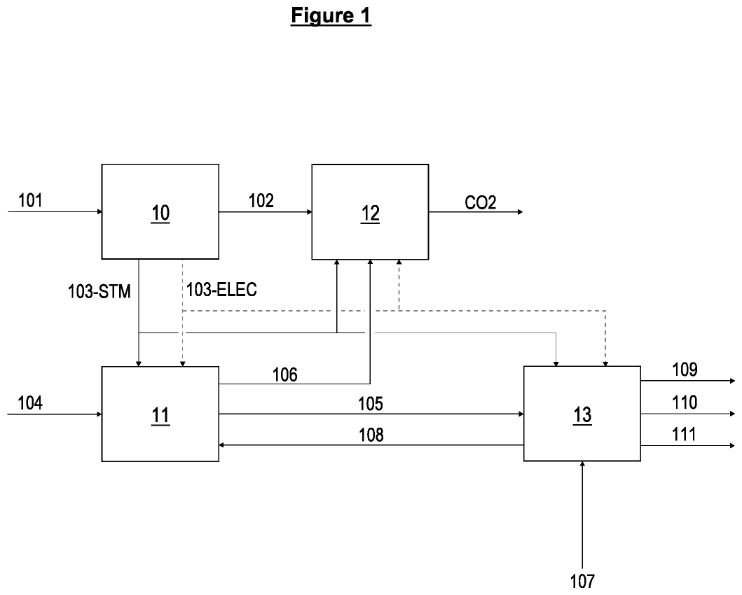

With reference to FIG. 1, a first embodiment of the present invention is depicted, in which low, neutral, and/or negative carbon intensity ethylene is produced by a steam cracking process wherein some of the required heating duty for the steam cracking process is provided by the combustion of low, neutral, and/or negative carbon intensity hydrogen produced by a reforming process, wherein the required energy is provided by a biomass power plant with carbon capture.

Biomass power plant 10 generates carbon negative energy in the form of electricity 103-ELEC and/or steam 103-STM. The energy produced from biomass power plant 10 is generated by either a Rankine cycle, a Brayton cycle, or an integrated gasification combined cycle which consists of both a Rankine cycle and Brayton cycle.

Where biomass power plant 10 comprises a Rankine cycle, energy is produced from the direct combustion of biomass feedstock 101 in a biomass boiler to generate steam. The generated steam from the biomass power plant can be sent to a Rankine cycle steam turbine to produce electricity 103-ELEC to provide power to hydrogen plant 11 and/or olefins production plant 13. Furthermore, steam 103-STM can be extracted from the turbine or sent directly to hydrogen plant 11 and/or olefins production plant 13.

Where biomass power plant 10 comprises a Brayton cycle, the biomass feedstock 101 is gasified to create a syngas product that is combusted as a fuel in a Brayton cycle gas turbine to produce electricity 103-ELEC.

Alternatively, biomass power plant 10 can be configured as an IGCC plant in which the biomass feedstock 101 is gasified to create a syngas product with or without steam generation. The syngas can be combusted as fuel in a Brayton cycle gas turbine to generate electricity 103-ELEC to provide power to hydrogen plant 11 and/or olefins production plant 13. Steam 103-STM can also be generated with a heat recovery steam generator (HRSG) on the gas turbine exhaust. This steam can be used in a Rankine cycle to generate additional electricity 103-ELEC. Steam 103-STM can also be used as process or dilution steam or as thermal energy in hydrogen plant 11 and/or olefins production plant 13. Additional uses of this carbon negative energy (103-ELEC and 103-STM) are discussed herein with respect to additional embodiments of the present invention.

Hydrocarbon feed 104 is reformed in hydrogen plant 11 by either autothermal reforming, steam methane reforming, or a combination of autothermal reforming and steam methane reforming to produce low, neutral, and/or negative carbon intensity hydrogen 105.

Hydrocarbon feed 104 to hydrogen plant 11 can be a variety of hydrocarbons including but not limited to natural gas, renewable natural gas, biogas, refinery off-gases, fuel gas, naphtha, and renewable naphtha.

Carbon dioxide 102 is captured from the biomass power plant flue gas stream and carbon dioxide 106 is captured from the hydrogen plant syngas stream and/or flue gas stream. The carbon dioxide streams 102 and 106 can be compressed in CO₂ compression unit 12 and sent to geologic sequestration or otherwise used external to the process.

Steam cracker feed 107, which comprises hydrocarbon molecules such as ethane, propane, butane, naphtha, and/or gas oil, is heated and “cracked” in the presence of steam to form smaller, unsaturated hydrocarbon molecules in olefins production plant 13. The cracked gas is quenched, compressed, treated, and separated into ethylene product 109, propylene product 110, and C₄+ hydrocarbons product 111.

The steam cracking process in olefins production plant 13 also produces an ethylene off gas (EOG) that comprises mostly methane and hydrogen. In all embodiments described herein, hydrogen 105 is sent to olefins production plant 13 as fuel to the steam cracker furnace where it is combusted to provide the required heating duty to the steam cracking process to reduce the CO₂ emissions generated from the combustion of a hydrocarbon fuel (e.g., ethylene off gas, natural gas, fuel gas) or reduce the CO₂e emissions associated with the production of electricity for an electric furnace.

The ethylene off gas can also be separated into hydrogen and methane. The hydrogen can be recycled and combined with hydrogen 105 as fuel to the stream cracking furnace. Methane 108 can be recycled to hydrogen plant 11 to be reformed to produce low, neutral, and/or negative carbon intensity hydrogen 105.

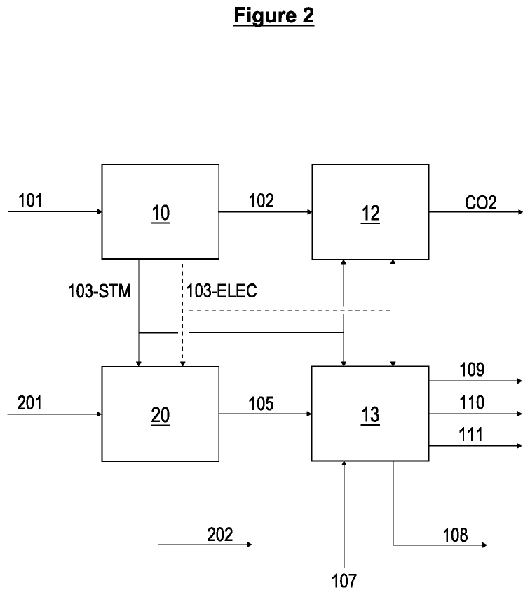

In a second embodiment of the present invention, FIG. 2 illustrates the production of low, neutral, and/or negative carbon intensity ethylene from a steam cracking process wherein some of the required heating duty for the steam cracking process is provided by the combustion of low, neutral, and/or negative carbon intensity hydrogen produced by electrolysis, wherein the required energy for the electrolysis process is provided by a biomass power plant with carbon capture.

Similar to FIG. 1, biomass power plant 10 generates carbon negative energy in the form of electricity 103-ELEC and/or steam 103-STM. The energy produced from biomass power plant 10 is generated by either a Rankine cycle, a Brayton cycle, or an integrated gasification combined cycle which consists of both a Rankine cycle and Brayton cycle.

As previously described in the first embodiment, electricity 103-ELEC can be produced from biomass power plant 10 to provide power to electrolysis unit 20. Furthermore, steam 103-STM can be sent directly to electrolysis unit 20 as process steam for high-temperature steam electrolysis (HTSE) or can be extracted from the turbine and sent as thermal energy to electrolysis unit 20. The use of steam 103-STM in electrolysis unit 20 decreases the amount of electricity required for the electrolysis process. Additional uses of this carbon negative energy (103-ELEC and 103-STM) are discussed herein with respect to additional embodiments of the present invention.

In electrolysis unit 20, water 201 is converted to a low, neutral, and/or negative carbon intensity hydrogen 105 and oxygen 202 through electrolysis using carbon negative energy in the form of electricity, 103-ELEC, and/or steam, 103-STM, from biomass power plant 10. As discussed above, the carbon negative energy produced from biomass power plant 10 can be combined with higher carbon intensity energy (not shown), including grid import power, to achieve a targeted carbon intensity of hydrogen 105.

Carbon dioxide 102 is captured from the biomass power plant flue gas stream. Carbon dioxide 102 can be compressed in CO₂ compression unit 12 and sent to geologic sequestration or can otherwise be used external to the process.

Steam cracker feed 107, which comprises hydrocarbon molecules such as ethane, propane, butane, naphtha, and/or gas oil, is heated and “cracked” in the presence of steam in olefins production plant 13. The cracked gas is quenched, compressed, treated, and separated into ethylene product 109, propylene product 110, and C₄+ hydrocarbons product 111.

The ethylene off gas can be separated into hydrogen and methane wherein the hydrogen can be recycled and combined with hydrogen 105 as fuel to the stream cracking furnace.

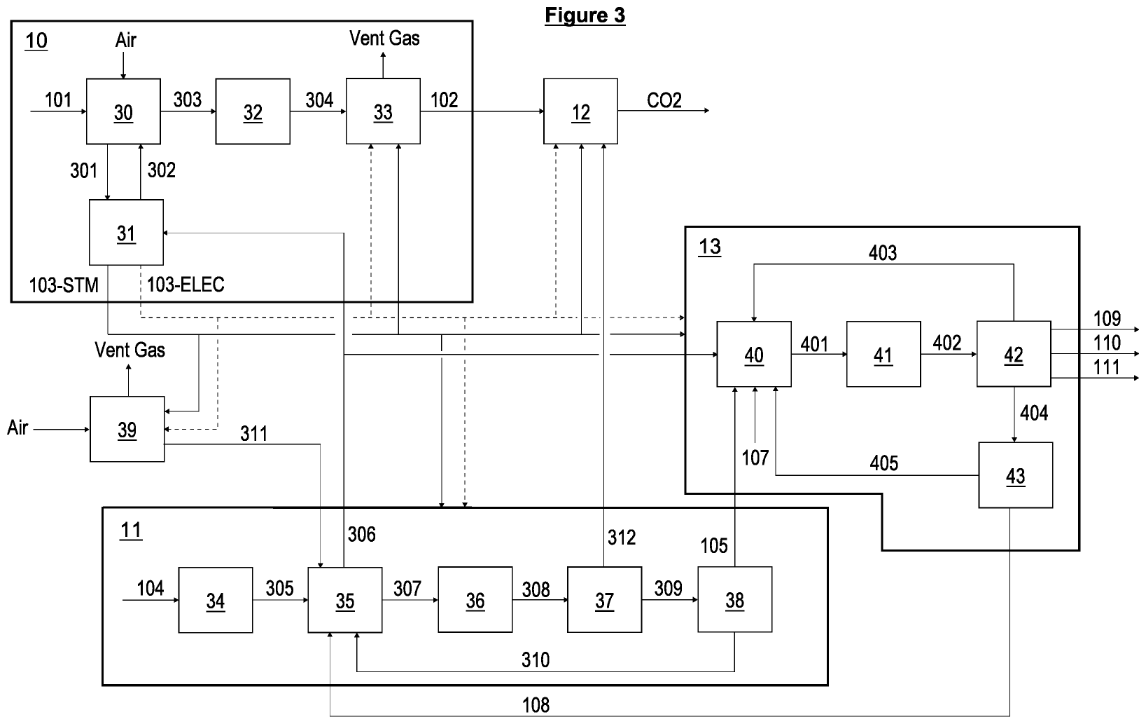

In a third embodiment of the present invention, FIG. 3 depicts the production of low, neutral, and/or negative carbon intensity ethylene from a steam cracking process utilizing hydrogen produced from an auto-thermal reformer (ATR) wherein some of the required energy for the hydrogen production is provided by biomass power plant 10, more specifically a Rankine cycle power plant, with carbon capture.

Biomass power plant 10 is depicted as a Rankine cycle power plant. Biomass feedstock 101 is combusted in boiler 30 to produce steam 301 from boiler feed water 302 that can be used to generate electricity 103-ELEC in steam turbine 31. Although not shown in FIG. 3, steam 103-STM can be extracted from steam turbine 31 to provide a source of thermal energy for process heating requirements, process steam to the steam methane reforming (SMR) reaction and/or water-gas shift (WGS) reaction, dilution steam to the steam cracking process, and/or steam drive energy to power the mechanical drives of compressors, pumps, and/or fans. Furthermore, electricity 103-ELEC and steam 103-STM can also be sent to hydrogen plant 11, CO₂ compression unit 12, and/or olefins production plant 13.

Flue gas 303 from boiler 30 is sent through air quality control system 32, where it passes through several emissions reduction technologies such as a pulse jet fabric filter, dry sorbent injection system, selective catalytic reduction (SCR), and carbon monoxide catalyst. These environmental train technologies are optional to treat flue gas 303 depending on the specific site needs, and would be well understood by a person having ordinary skill in the art.

Also not shown in FIG. 3, electricity 103-ELEC and steam 103-STM from biomass power plant can be generated by the gasification of the biomass feedstock. The gasification of the biomass in the presence of air, steam, or oxygen results in a syngas product consisting of mostly carbon monoxide and hydrogen. Any impurities produced in the syngas are removed in a syngas clean-up step. The syngas can be combusted in a gas turbine to produce electricity 103-ELEC. Optionally, a HRSG can be included on the turbine exhaust to produce steam. This steam can be used to generate additional electricity in a steam turbine or can be sent directly to the hydrogen plant for use as thermal energy for process heating requirements, process steam to either the SMR reaction and/or WGS reaction, dilution steam to the steam cracking process, and/or steam drive energy to power the mechanical drives of compressors, pumps and/or fans. The exhaust from the HRSG can be sent to a post-combustion carbon capture unit to reduce CO₂e emissions.

The treated flue gas 304 is then sent to PCC unit 33. The PCC technology employed for carbon capture can include a cryogenic process or an amine-based solution. Captured carbon dioxide 102 can be compressed in CO₂ compression unit 12 and sent to geologic sequestration or can otherwise be used external to the process.

Hydrocarbon feed 104 goes through feed purification 34 to remove any sulfur compounds and other contaminants. Hydrocarbon feed 104 can also be compressed depending on the upstream operating pressure of the feed.

Treated hydrocarbon feed 305 is then sent to reformer 35. Treated hydrocarbon feed 305 is preheated prior to reforming. There are multiple options for preheating treated hydrocarbon feed 305, including but not limited to a fired preheater and/or a gas heated reformer (GHR). Depending on the desired carbon dioxide recovery, a PCC unit can also be included on any fired preheater to capture the CO₂ from the flue gas (not shown). Where the configuration includes a standalone GHR, it is well understood that no flue gas would be generated.

In reformer 35, the preheated and treated hydrocarbon feed undergoes an endothermic steam methane reforming reaction and an exothermic partial oxidation (PDX) reaction. The heat produced by the PDX reaction is used in the steam methane reforming reaction to generate syngas. In an ATR, the hydrocarbons are partially oxidized, in the presence of high purity oxygen 311 generated by air separation unit (ASU) 39 to form syngas 307, which consists of carbon monoxide and hydrogen.

Syngas 307 generated by the ATR, which operates at elevated temperatures due to the endothermic steam methane reforming reaction, can be utilized to generate steam 306 from boiler feed water in reformer 35. Steam can also be generated from the preheater in reformer 35. Steam 306 can be sent to steam turbine 31 at biomass power plant 10 to generate additional electricity. Alternatively, steam 306 produced in reformer 35 can be used as dilution steam in cracking unit 40, or can be sent to a separate steam turbine located in either hydrogen plant 11 or olefins production plant 13 to create additional electricity, or can be used external to the process (e.g. district heating).

Syngas 307 from reformer 35 is sent to water-gas shift reactor 36 where carbon monoxide and water in the syngas undergo a WGS reaction to form carbon dioxide and hydrogen.

The hydrogen and carbon dioxide product 308 from WGS reactor 36 can be sent to syngas carbon capture unit 37 where the carbon dioxide is separated using either a cryogenic process or an amine-based solution. Captured carbon dioxide 312 can be combined with carbon dioxide 102 from biomass power plant 10 and can be compressed in CO₂ compression unit 12 and sent to geologic sequestration or can otherwise be used external to the process.

Hydrogen 309 from syngas carbon capture unit 37 is sent to hydrogen purification unit 38. Commonly, hydrogen purification unit 38 consists of a pressure swing adsorption (PSA) system, but other alternative technologies known in the art, such as membranes, can also be utilized to achieve the desired product composition for hydrogen 105.

Tail gas/reject gas 310 from hydrogen purification unit 38, which consists of mostly hydrogen, can be used as fuel for either the fired preheater in reformer 35, biomass boiler 30 (not shown), or in a utility boiler (not shown). Although not shown, hydrogen 309 from syngas carbon capture unit 37 can be sent directly as fuel to the steam cracking furnace in cracking unit 40 and thus minimize or eliminate the need for hydrogen purification unit 38.

Steam cracker feed 107, which can be comprised of ethane, propane, butane, naphtha, gas oil or a combination thereof, is sent to olefins production plant 13 where it is combined with dilution steam. The dilution steam can be provided from the quench system in cracking unit 40, steam 306 from hydrogen plant 11, and/or 103-STM from biomass power plant 10.

Steam cracker feed 107 in the presence of dilution steam is heated to elevated temperatures in the steam cracking furnace to generate cracked gas 401, which consists of ethylene, propylene, and various by-products. Cracked gas 401 is quenched to control the cracking reaction and thermal energy from cracked gas 401 is used to produce steam that is commonly used as dilution steam and/or steam drive energy to power the mechanical drive (e.g., turbines) of the gas compressors.

Cracked gas 401 is sent to compression and drying 41, where impurities such as carbon dioxide, sulfur, and water are removed to produce treated cracked gas 402.

Treated cracked gas 402 is sent to product recovery section 42. As would be well understood by a person having ordinary skill in the art, the components of treated cracked gas 402 are separated into commercial chemical products by a demethanizer, deethanizer, C₂ splitter, depropanizer, C₃ splitter, and a debutanizer in recovery section 42.

In one typical configuration for recovery section 42, treated cracked gas 402 is sent to the demethanizer. Ethylene off gas 404, which consist of hydrogen and methane, is recovered from the overhead of the demethanizer column.

The bottoms of the demethanizer column, consisting of C₂+ hydrocarbons, are sent to the deethanizer. The C₂ hydrocarbons are separated from the cracked gas in the overhead of the deethanizer column and are then sent to a C₂ splitter where ethylene product 109 is separated and recovered from the overhead of the C₂ splitter. The bottoms of the C₂ splitter, which consist of ethane, are recycled back to cracking unit 40.

The deethanizer bottoms, consisting of C₃+ hydrocarbons, are sent to the depropanizer. The C₃ hydrocarbons are separated from the cracked gas in the overhead of the depropanizer column are then sent to a C₃ splitter where propylene product 110 is separated and recovered from the overhead of the C₃ splitter. The bottoms of the C₃ splitter, which consist of mostly propane, are recycled back to cracking unit 40.

Ethane and propane recycle 403 is combined with steam cracker feed 107 and dilution steam in cracking unit 40 where it is heated and cracked in the steam cracking furnace.

The depropanizer bottoms, consisting of C₄+ hydrocarbons are sent to the debutanizer where butanes are separated and recovered from the overhead of the column. The debutanizer bottoms consist of C₅+ hydrocarbons.

Ethylene off gas 404 from the demethanizer is sent to hydrogen recovery unit 43 where the recovered methane 108 and hydrogen 405 are separated. Hydrogen recovery unit 38 consists of a pressure swing adsorption PSA system, but other alternative technologies known in the art, such as cryogenic separation and/or membranes, can also be utilized to achieve the desired product composition for hydrogen 405.

Hydrogen 405 can be combined with hydrogen 105 as a fuel to the steam cracker furnace, where it is combusted to provide the required heating duty to the steam cracking process to reduce the CO₂ emissions generated from the combustion of a hydrocarbon fuel or reduce the CO₂e emissions associated with the production of electricity for an electric furnace.

Recovered methane 108 can be recycled back to hydrogen plant 11 where it can be combined with hydrocarbon feed 104 and reformed to produce low, neutral, and/or negative carbon intensity hydrogen 105.

Although not shown, ethylene off gas 404 can be recycled back the hydrogen plant 11, where the methane will be reformed to produce additional low, neutral, and/or negative carbon intensity hydrogen 105. The hydrogen in the ethylene off gas will pass through the reformer and comingle with hydrogen 105 as fuel to the steam cracker furnace.

Although not shown, ethylene off gas 404 can be sent as a fuel to the steam cracker furnace in cracking unit 40, fired preheater in reformer 35, biomass boiler 30, and/or utility boiler (not shown).

The use of carbon negative energy from biomass power plant 10, can also be supplied to olefins production plant 13 in four forms to offset the CO₂e emissions associated with the production of ethylene and/or CO₂e emissions associated with import grid power: (1) electricity 103-ELEC that can be used to provide power to the steam cracking furnace, compressor, pump, and/or fan motors, auxiliary equipment, instrumentation, and controls; (2) dilution steam 103-STM for the steam cracking process; (3) thermal energy in the form of steam 103-STM for process heating requirements; and/or (4) steam drive energy to power the mechanical drive (e.g., turbines) of the compressors, pumps, and/or fans.

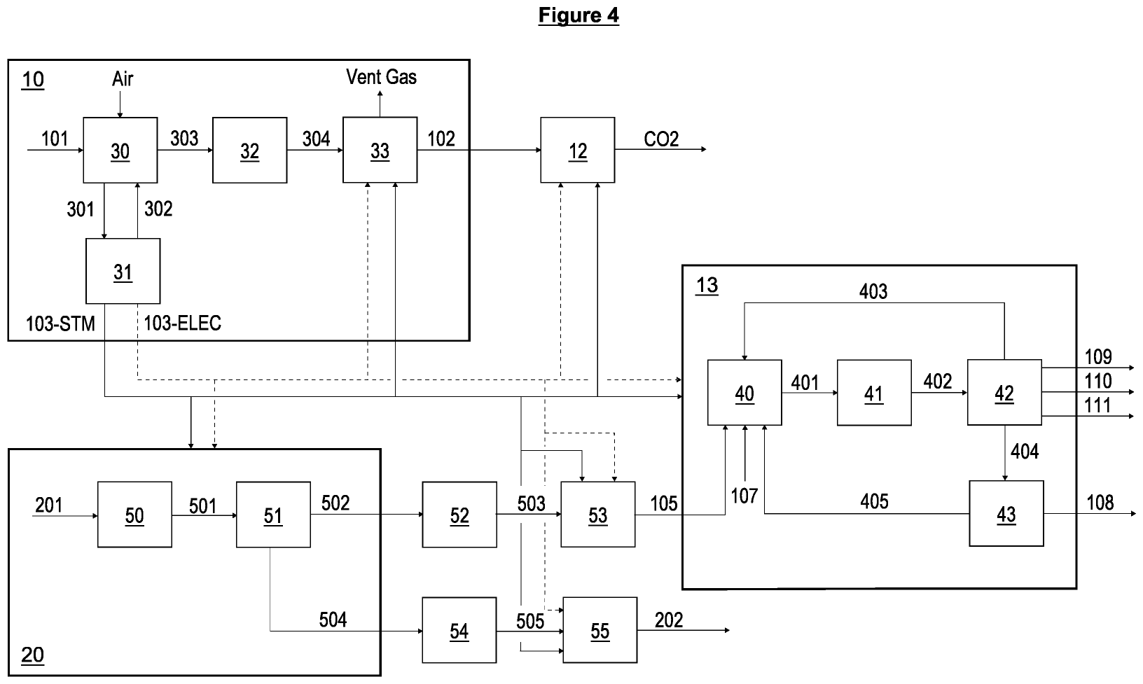

In a fourth embodiment of the present invention, FIG. 4 illustrates the production of low, neutral, and/or negative carbon intensity ethylene from a steam cracking process utilizing hydrogen produced from electrolysis wherein some of the required energy for the electrolysis process is provided by biomass power plant 10, more specifically a Rankine cycle power plant, with carbon capture.

FIG. 4 differs from FIG. 3 in the method of hydrogen production; biomass power plant 10, CO₂ compression unit 12, and olefins production plant 13 are similar to those depicted in FIG. 3.

Raw water 201 is sent to water treatment unit 50 in electrolysis unit 20 to remove impurities, such as particulates, organic compounds, and/or mineral salts to produce treated or deionized water 501. Depending on the quality of the raw water, different technologies may be required to achieve the desired treated water quality, as would be understood by a person having ordinary skill in the art. Treated or deionized water 501 is sent to electrolyzer 51.

Electrolyzer 51 consists of multiple electrolytic stacks that contain a cathode and an anode that are separated by a membrane. Electrolysis occurs when an electric current is applied across the electrolyte, thereby splitting water into hydrogen and oxygen.

Where electrolyzer 51 is comprised of alkaline water electrolysis (AWE), water reacts with the cathode of the electrolytic cell stack to form hydrogen and negatively charged hydroxide ions.

Where electrolyzer 51 is comprised of polymer electrolyte membrane (PEM), water reacts with the anode of the electrolytic cell stack to form oxygen and positively charged hydrogen ions. The positively charged hydrogen ions pass through the electrolyte membrane and combine at the cathode to form hydrogen.

Where electrolyzer 51 is comprised of a solid oxide electrolyzer, steam at elevated temperatures reacts with the cathode to form hydrogen and negatively charged oxygen ions. The negatively charged oxygen ions pass through the solid membrane and combine at the anode to form oxygen.

Hydrogen produced in electrolyzer 51 is sent to a water separator (not shown) to remove water. Depending on the desired moisture content, the hydrogen can also be sent to a dryer system. Temperature swing adsorption (TSA) systems are commonly used to achieve the desired moisture content of hydrogen product 502, but other alternative technologies known in the art can be utilized.

Hydrogen 502 is sent to hydrogen storage 52, and stored hydrogen 503 can be compressed in hydrogen compression unit 53. Hydrogen 105 is sent to olefins production plant 13 as fuel to the steam cracker furnace.

Oxygen produced in electrolyzer 51 is sent to a water separator (not shown) to remove water. Oxygen 504 is sent to oxygen storage 54, and stored oxygen 505 can be compressed in oxygen compression unit 55. Although not shown, where biomass power plant 10 comprises a Rankine cycle power plant with an oxy-fired boiler, oxygen 504 produced by electrolysis unit 20 can be utilized in boiler 30.

Although not shown, in FIG. 4, steam 301 can also be sent directly to electrolysis unit 20 for use as process steam for HTSE, thermal energy for process heating requirements, and/or steam drive energy to power mechanical drive for rotating equipment.

The illustration of olefins production plant 13 in FIG. 4 differs slightly from FIG. 3. Methane 108 from hydrogen recovery unit 38 can be combined with biomass feedstock 101 and be combusted in boiler 30 (not shown) and/or can be utilized external to the process.

Carbon negative energy from biomass power plant 10 can be supplied to electrolysis unit 20 in four forms: (1) electricity 103-ELEC that can be used to provide power to compressor, pump, and/or fan motors, auxiliary equipment, instrumentation, and controls; (2) process steam for high-temperature steam electrolysis, including but not limited to the use of solid oxide electrolytic cells (SOEC); (3) thermal energy in the form of steam 103-STM for process heating requirements and/or (4) steam drive energy to power the mechanical drive (e.g., turbines) of the compressors, pumps, and/or fans.

Although not shown in the illustrative embodiments described above, hydrogen can also be produced from a combination of reforming methane 108 from the ethylene off gas and through electrolysis of water. By utilizing carbon negative electricity and/or steam (103-ELEC and 103-STM) to provide energy to hydrogen plant 11, electrolysis unit 20, CO₂ compression unit 12, and/or olefins production plant 13, the carbon intensity of ethylene product 109 can be reduced to less than 0.0 CO₂e/kg C₂H₄.

In an illustrative embodiment of the present invention, Table 1 demonstrates the well-to-gate carbon intensity of ethylene produced by various steam cracker configurations.

TABLE 1

| | | Steam | Steam | Steam | Steam | Steam |
|---|---|---|---|---|---|---|
| | | Cracker | Cracker | Cracker | Cracker | Cracker |
| Configuration | Unit | Configuration #1 | Configuration #2 | Configuration #3 | Configuration #4 | Configuration #5 |
| Steam Cracker Feed | wt % | 76% Ethane | 76% Ethane | 76% Ethane | 76% Ethane | 76% Ethane |
| Composition | | 16% Propane | 16% Propane | 16% Propane | 16% Propane | 16% Propane |
| | | 3% Butane | 3% Butane | 3% Butane | 3% Butane | 3% Butane |
| | | 6% Naphtha | 6% Naphtha | 6% Naphtha | 6% Naphtha | 6% Naphtha |
| Steam Cracking Furnace Fuel | — | EOG | EOG | Produced | Produced | Produced |
| | | | | Hydrogen < −0.1 | Hydrogen < −0.2 | Hydrogen < −10 |
| | | | | kg CO₂e/kg H₂ | kg CO₂e/kg H₂ | kg CO₂e/kg H₂ |
| Post-Combustion Capture on | Y/N | N | Y | N | N | N |
| Steam Cracking Furnace | | | | | | |
| Included? | | | | | | |
| Reforming with Biomass | Y/N | N | N | Y | Y | Y |
| Power Plant Included? | | | | | | |
| Electrolysis with Biomass | Y/N | N | N | N | N | Y |
| Power Plant Included? | | | | | | |
| CO₂e from with Combustion | kg CO₂e/ | 0.37 | 0.03 | 0.00 | −0.18 | −0.56 |
| | kg C₂H₄ | | | | | |
| CO₂e from Non-Combustion | kg CO₂e/ | 0.05 | 0.05 | 0.05 | 0.05 | 0.05 |
| | kg C₂H₄ | | | | | |
| CO₂e from Upstream | kg CO₂e/ | 0.51 | 0.64 | 0.51 | 0.51 | 0.51 |
| Emissions | kg C₂H₄ | | | | | |
| Ethylene Carbon Intensity | kg CO₂e/ | 0.93 | 0.72 | 0.56 | 0.38 | 0.00 |
| | kg C₂H₄ | | | | | |

The steam cracker feed composition in Table 1 represents a blended mass fraction of the average feedstock for ethylene production as outlined from the Oil and Gas Journal's 2015 International Survey of Ethylene from Steam Crackers as presented in GREET's 2022 release.

The ratio of naphtha to natural gas liquids (NGL), which contains ethane, propane, and/or butanes, has a meaningful impact on the well-to-gate carbon intensity of the ethylene product. Based on steam cracker configuration #1, outlined in Table 1 above, a feedstock that is 100% naphtha results in well-to-gate ethylene carbon intensity of 1.16 kg CO₂e/kg C₂H₄ whereas a feedstock that is 100% ethane results in a well-to-gate ethylene carbon intensity of 0.88 kg CO₂e/kg C₂H₄.

As shown in Table 1, the well-to-gate carbon intensity of the ethylene product can be reduced with the inclusion of a PCC and can be further reduced by utilizing low, neutral, and/or negative carbon intensity hydrogen as fuel to the stream cracking furnace, in accordance with certain teachings of the present invention.

In a second illustrative embodiment of the present invention, Table 2 demonstrates the well-to-gate carbon intensity of a both the ethylene and propylene product based on the three main methods of allocating emissions within the steam cracker as modeled in GREET (i.e., mass, energy, and value allocation).

TABLE 2

| | | Mass | Energy | Value |
|---|---|---|---|---|
| | Units | Allocation | Allocation | Allocation |
| Ethylene | kg CO₂e/kg C₂H₄ | 0.93 | 0.94 | 0.95 |
| Product | | | | |
| Propylene | kg CO₂e/kg C₃H₆ | 0.93 | 0.91 | 0.82 |
| Product | | | | |

As shown in Table 2, the well-to-gate carbon intensity of the co-product, propylene, varies slightly between allocation methods, but is tied to the overall steam cracker emissions. Therefore, the well-to-gate carbon intensity of the propylene product can also be reduced in accordance with certain teachings of the present invention.

In yet another embodiment of the present invention, a method for producing an ethylene product having a well-to-gate carbon intensity preferably less than about 0.6 kg CO₂e/kg C₂H₄, more preferably less than about 0.4 kg CO₂e/kg C₂H₄, and most preferably less than about 0.0 kg CO₂e/kg C₂H₄, is provided. Also included is a method for producing a propylene product having a well-to-gate carbon intensity preferably less than about 0.6 kg CO₂e/kg C₃H₆, more preferably less than about 0.4 kg CO₂e/kg C₃H₆, and most preferably less than about 0.0 kg CO₂e/kg C₃H₆. The method comprises the steps of converting a hydrocarbon feedstock to an unsaturated hydrocarbon stream through a steam cracking process in an olefins production plant; combusting hydrogen to provide at least some of the heating duty to the steam cracking process, wherein the hydrogen has a carbon intensity preferably less than about 1.0 kg CO₂e/kg H₂, more preferably less than less than about 0.45 kg CO₂e/kg H₂, and most preferably less than about 0.0 kg CO₂e/kg H₂, wherein the hydrogen is produced using a hydrogen production process; providing at least some, and preferably substantially all, of the required energy for the hydrogen production process from a biomass power plant; and processing the unsaturated hydrocarbon stream to recover the olefin product. The hydrocarbon feedstock for the olefins production plant is selected from ethane, propane, butane, naphtha, gas oils, or a combination thereof. The energy produced by the biomass power plant is selected from one or more of: (a) electricity generated from work produced by a Rankine cycle, Brayton cycle, or integrated gasification combine cycle; (b) steam that can be used as process steam or dilution steam; (c) steam that can be used as thermal energy; and (d) steam that can be used to power a mechanical drive. One or more gas streams containing carbon dioxide from the biomass power plant may be processed in one or more carbon capture units to reduce CO₂e emissions. The hydrogen production process may comprise converting a hydrocarbon feedstock to hydrogen through a reforming process, or may comprise the electrolysis of water, or both. At least some, and preferably substantially all, of the required electricity and steam energy for olefins production plant is provided from the biomass power plant. At least some of the required heating duty for the combusting step is provided by combusting recycled hydrogen that is separated from the unsaturated hydrocarbon stream. Methane is separated from the unsaturated hydrocarbon stream, wherein the methane is combusted in the biomass power plant. Furthermore, an offgas stream comprising methane may be separated from the unsaturated hydrocarbon stream, wherein the offgas stream is combusted in one or more of the hydrogen production process, the steam cracking process, or an auxiliary boiler. One or more flue gas streams containing carbon dioxide from the hydrogen production process, the steam cracking process, or an auxiliary boiler may be processed in one or more carbon capture units to reduce CO₂e emissions, wherein at least some of the energy for the carbon capture units is provided from the biomass power plant. Furthermore, the carbon capture unit comprises compressing or pumping the captured carbon dioxide therefrom, wherein at least some of the required energy for compressing or pumping the carbon dioxide is provided from the biomass power plant. The olefins production plant comprises an ethylene refrigeration system, wherein at least some of the required energy for the ethylene refrigeration system is provided from the biomass power plant. The olefins production plant comprises a propylene refrigeration system, wherein at least some of the required energy for the ethylene refrigeration system is provided from the biomass power plant.

Where hydrogen is produced in a hydrogen production process comprising converting a hydrocarbon feedstock to hydrogen through a reforming process, methane is separated from the unsaturated hydrocarbon stream, wherein the separated methane is recycled to the reforming process to produce additional hydrogen, wherein at least some of the required heating duty for the combusting step is provided by combusting the additional hydrogen. The carbon dioxide from the hydrogen production process is captured in one or more carbon capture units to reduce CO₂e emissions, wherein at least some, and preferably substantially all, of the energy for the one or more carbon capture units is provided from the biomass power plant. The tail gas from the reforming process is combusted in one or more of the biomass power plant, the hydrogen production process, the steam cracking process, or an auxiliary boiler. The hydrocarbon reforming process is selected from autothermal reforming, steam methane reforming, or a combination of autothermal reforming and steam methane reforming. Furthermore, the hydrogen production process comprises an air separation unit to provide high purity oxygen to the hydrogen production process, wherein at least some of the required energy for the air separation unit is provided from the biomass power plant.

Where hydrogen is produced in in a hydrogen production process comprising electrolysis of water, the electrolysis process is selected from polymer electrolyte membrane electrolysis, alkaline electrolysis, solid oxide electrolysis, or a combination thereof. Furthermore, the biomass power plant comprises oxy-combustion of the biomass feedstock, wherein at least some of the required oxygen required for the oxy-combustion is provided by the electrolysis process. The electrolysis process also comprises compressing the hydrogen product therefrom, wherein at least some of the required energy for the hydrogen compressor is provided from the biomass power plant.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings therein. It is therefore evident that the particular embodiments disclosed above may be altered or modified and all such variations are considered within the scope and spirit of the present invention.

What is claimed is:

1. A method for producing an olefin product, comprising converting a hydrocarbon feedstock to an unsaturated hydrocarbon stream through a steam cracking process in an olefins production plant;
combusting hydrogen to provide at least some of the heating duty to the steam cracking process, wherein the hydrogen has a carbon intensity less than about 1.0 kg CO₂e/kg H₂; wherein the hydrogen is produced using a hydrogen production process;
providing at least some of the required energy for the hydrogen production process from a biomass power plant; and
processing the unsaturated hydrocarbon stream to recover the olefin product, wherein the olefin product comprises ethylene having a well-to-gate carbon intensity less than 0.6 kg CO₂e/kg C₂H₄.

2. The method of claim 1, wherein the olefin product comprises propylene having a well-to-gate carbon intensity less than 0.6 kg CO₂e/kg C₃H₆.

3. The method of claim 1, wherein the energy produced by the biomass power plant is selected from one or more of:
(a) electricity generated from work produced by a Rankine cycle, Brayton cycle, or integrated gasification combine cycle;
(b) steam that can be used as process steam or dilution steam;
(c) steam that can be used as thermal energy; and
(d) steam that can be used to power a mechanical drive.

4. The method of claim 1, further comprising processing one or more gas streams containing carbon dioxide from the biomass power plant in one or more carbon capture units to reduce CO₂e emissions.

5. The method of claim 1, wherein the hydrogen is produced in a hydrogen production process comprising converting a hydrocarbon feedstock to hydrogen through a reforming process.

6. The method of claim 1, wherein the hydrogen is produced in a hydrogen production process comprising electrolysis of water.

7. The method of claim 1, wherein the hydrogen is produced from both a reforming process and electrolysis of water.

8. The method of claim 1, further comprising providing substantially all of the required energy for the hydrogen production process from a biomass power plant.

9. The method of claim 1, wherein at least some of some of the required heating duty for the combusting step is provided by combusting recycled hydrogen that is separated from the unsaturated hydrocarbon stream.

10. The method of claims 5, wherein methane is separated from the unsaturated hydrocarbon stream, wherein the separated methane is recycled to the reforming process to produce additional hydrogen, and wherein at least some of some of the required heating duty for the combusting step is provided by combusting the additional hydrogen.

11. The method of claim 1, further comprising providing at least some of the required electricity and steam energy for olefins production plant from the biomass power plant.

12. The method of claim 1, further comprising providing substantially all the required electricity and steam energy for the olefins production plant from the biomass power plant.

13. The method of claims 5, further comprising capturing the carbon dioxide from the hydrogen production process in one or more carbon capture units to reduce CO₂e emissions, wherein at least some of the energy for the one or more carbon capture units is provided from the biomass power plant.

14. The method of claim 1, wherein the hydrocarbon feedstock for the olefins production plant is selected from ethane, propane, butane, naphtha, gas oils, or a combination thereof.

15. The method of claims 5, wherein the hydrocarbon reforming process is selected from autothermal reforming, steam methane reforming, or a combination of autothermal reforming and steam methane reforming.

16. The method of claim 6, wherein the electrolysis process is selected from polymer electrolyte membrane electrolysis, alkaline electrolysis, solid oxide electrolysis, or a combination thereof.

17. The method of claims 5, wherein the hydrogen production process produces a tail gas that is combusted in one or more of the biomass power plant, the hydrogen production process, the steam cracking process, or an auxiliary boiler.

18. The method of claim 1, wherein methane is separated from the unsaturated hydrocarbon stream, wherein the methane is combusted in the biomass power plant.

19. The method of claims 5, wherein an offgas stream comprising methane is separated from the unsaturated hydrocarbon stream, wherein the offgas stream is combusted in one or more of the hydrogen production process, the steam cracking process, or an auxiliary boiler.

20. The method of claim 6, wherein an offgas stream comprising methane is separated from the unsaturated hydrocarbon stream, wherein the offgas stream is combusted in one or more of the steam cracking process or an auxiliary boiler.

21. The method of claims 19, further comprising processing one or more flue gas streams containing carbon dioxide from the hydrogen production process, the steam cracking process, or an auxiliary boiler in one or more carbon capture units to reduce CO₂e emissions, wherein at least some of the energy for the carbon capture units is provided from the biomass power plant.

22. The method of claim 1, wherein the olefins production plant comprises compressing the unsaturated hydrocarbon stream, wherein at least some of the required energy for the compressing step is provided from the biomass power plant.

23. The method of claim 1, wherein the olefins production plant comprises separating the unsaturated hydrocarbon stream in one or more distillation columns, wherein at least some of the required energy for separating step is provided from the biomass power plant.

24. The method of claim 23, wherein at least some of the required energy for separating step comprises electricity for one or more of column bottoms, overhead, reflux, and product pump motors.

25. The method of claim 23, wherein at least some of the required energy for separating step comprises electricity for one or more of column overhead air cooler and product air cooler fan motors.

26. The method of claim 23, wherein at least some of the required energy for separating step comprises steam used to power mechanical drives for one or more of compressors, pumps, and fans.

27. The method of claim 23, wherein at least some of the required energy for separating step comprises steam used as thermal energy for one or more column reboilers.

28. The method of claim 1, wherein the olefins production plant comprises an ethylene refrigeration system, wherein at least some of the required energy for the ethylene refrigeration system is provided from the biomass power plant.

29. The method of claim 1, wherein the olefins production plant comprises a propylene refrigeration system, wherein at least some of the required energy for the propylene refrigeration system is provided from the biomass power plant.

30. The method of claim 6, wherein the biomass power plant comprises oxy-combustion of the biomass feedstock, wherein at least some of the required oxygen required for the oxy-combustion is provided by the electrolysis process.

31. The method of claims 5, wherein the hydrogen production process comprises an air separation unit to provide high purity oxygen to the hydrogen production process, wherein at least some of the required energy for the air separation unit is provided from the biomass power plant.

32. The method of claim 6, wherein the electrolysis process comprises compressing the hydrogen product therefrom, wherein at least some of the required energy for the hydrogen compressor is provided from the biomass power plant.

33. The method of claims 4, wherein the carbon capture unit comprises compressing or pumping the captured carbon dioxide therefrom, wherein at least some of the required energy for compressing or pumping the carbon dioxide is provided from the biomass power plant.

34. The method of claim 1, wherein the hydrogen has a carbon intensity less than about 0.45 kg CO₂e/kg H₂.

35. The method of claim 1, wherein the hydrogen has a carbon intensity less than about 0.0 kg CO₂e/kg H₂.

36. The method of claim 1, wherein the ethylene has a well-to-gate carbon intensity less than about 0.4 kg CO₂e/kg C₂H₄.

37. The method of claim 1, wherein the ethylene has a well-to-gate carbon intensity less than about 0.0 kg CO₂e/kg C₂H₄.

38. The method of claim 2, wherein the propylene has a well-to-gate carbon intensity less than about 0.4 kg CO₂e/kg C₃H₆.

39. The method of claim 2, wherein the propylene has a well-to-gate carbon intensity less than about 0.0 kg CO₂e/kg C₃H₆.
